(19) United States
(12) Patent Application Publication
(10) Pub. No.: US 2023/0149944 A1
(43) Pub. Date: May 18, 2023

(54) ICE CORE ANALYSIS OF END PIT LAKES

(71) Applicant: SYNCRUDE CANADA LTD. in trust for the owners of, Calgary (CA)

(72) Inventors: Barry BARA, Edmonton (CA); Richard PAPROSKI, Edmonton (CA)

(21) Appl. No.: 18/055,543

(22) Filed: Nov. 15, 2022

Related U.S. Application Data
(60) Provisional application No. 63/279,988, filed on Nov. 16, 2021.

(51) Int. Cl. B03B 9/02; E21B 43/24

(52) U.S. Cl. CPC B03B 9/02; E21B 43/2405; B03D 2203/006

(57) ABSTRACT

A method for determining the source of a surface bitumen sheen in an end pit lake formed by water capping of oil sand tailings is provided comprising quantifying bitumen flux using ice core samples of the end pit lake. The high areas of bitumen flux indicate the areas of the end pit lake that should be dredged for reclamation of the end pit lake to prevent the formation of hydrocarbon sheen on the surface of the end pit lake. A method for evaluating reclamation of an end pit lake is also provided.

### TECHNICAL FIELD

The following relates generally to methods for monitoring end pit lakes for surface bitumen to aid in their reclamation. The methods are particularly useful for reclaiming end pit lakes formed by water capping of tailings, more particularly, but not limited to, fluid fine tailings (FFT) that are produced during oil sand extraction processes.

### BACKGROUND

Oil sand generally comprises water-wet sand grains held together by a matrix of viscous heavy oil or bitumen. Bitumen is a complex and viscous mixture of large or heavy hydrocarbon molecules that contain a significant amount of sulfur, nitrogen and oxygen. The extraction of bitumen from oil sand using hot/warm water processes yields large volumes of tailings composed of fine silts, clays and residual bitumen, which have traditionally been contained in a tailings pond. Mineral fractions with a particle diameter less than 44 microns are referred to as “fines.” These fines are typically quartz and clay mineral suspensions, predominantly kaolinite and illite.

The general term of fluid fine tailings (FFT) encompasses the spectrum of tailings that are produced as a result of the extraction of bitumen from oil sand using hot/warm water processes. FFT behaves as a fluid colloidal-like material. The fact that FFT behaves as a fluid and has very slow consolidation rates limits options to reclaim tailings ponds. Water capping tailings technology is a cost effective means to reclaim FFT and to integrate an aquatic landform in the closure landscape. Water capping tailings technology includes placing water, generally oil sand process water, over tailings material deposited below grade in an end pit (typically in a mined out area) to create a relatively shallow lake in the closure landscape. Eventually, the lake will evolve towards a viable lake that can sequester the FFT and support a developing aquatic ecosystem.

Sequestering FFT below a water cap in an end pit is a cost effective means to reclaim FFT. However, it was discovered by the present applicant that there might be problems with the acceptance of such an end pit lake as a viable end pit lake due to the presence of a hydrocarbon sheen on the water surface. Thus, there is a need in the industry for a viable hydrocarbon mitigation strategy to address the hydrocarbon sheen. In particular, there is a need in the industry for quantitative measurement methods for determining the source of these bitumen sheens so that the appropriate intervention can occur to prevent these sheens from developing.

### SUMMARY

Broadly stated, in one aspect, a method for determining a source of a surface bitumen sheen in an end pit lake formed by water capping of oil sand tailings is provided, comprising:
using an ice coring device to obtain an ice core sample having an area from the end pit lake;
melting the ice core and measuring a bitumen content in grams in the ice core sample; and
quantifying a bitumen flux by dividing the grams of bitumen by the area of the core divided by a time period between initial ice formation and the time the ice core was obtained.

In one embodiment, a gas content of the ice core is quantified using a computed tomography (CT) system to measure a gas flux in the end pit lake for monitoring microbial activity in the end pit lake over time. In one embodiment, a medical CT scanner is used, as the density of ice is very close to that of a human body for which the medical scanner is optimized. In one embodiment, the gas content is measured as milliliters of gas per ice core area. In one embodiment, gas flux is determined by the milliliters of gas divided by the area of the core and the time period between initial ice formation and the time the ice core was obtained.

In one embodiment, the bitumen content in the ice core is measured by melting the ice core and using the Dean & Stark Soxhlet extraction method to measure the grams of bitumen. In one embodiment, the amount of bitumen is measured as grams of bitumen per ice core area. In one embodiment, the melted ice core is passed through a Dean & Stark extraction thimble such that the bitumen droplets and fine solids are retained in the thimble while the melted water that passes through the thimble is collected in a separate container to be optionally measured gravimetrically. Only the portion of the melted ice core sample that is retained in the thimble is analysed by Dean & Stark Soxhlet extraction. This avoids the time-consuming step of boiling the melted water that passes through the thimble, which can exceed 5 kg in weight, during Dean & Stark analysis.

In one embodiment, the oil sand tailings comprises fluid fine tailings (FFT).

In another aspect, a method for evaluating end pit lake reclamation is provided, comprising:
using an ice coring device to obtain an ice core sample from the end pit lake and an ice core sample from a fresh water body;
melting the end pit lake and fresh water body ice cores and extracting the hydrocarbons therein using toluene to obtain an end pit lake toluene extract and a fresh water body toluene extract;
determining an Ultraviolet-Visible absorbance spectrum of the end pit lake toluene extract and an Ultraviolet-Visible absorbance spectrum of the fresh water body toluene extract; and
comparing the two spectra,
   whereby, when the end pit lake spectrum is substantially the same as the fresh water spectrum, it is an indication that the end pit lake is developing the capability to support aquatic life forms.

In one embodiment, the two spectra are compared to determine whether there is a peak at a wavelength of 670 nm in each of the spectra indicating the presence of plankton.

### BRIEF DESCRIPTION OF THE DRAWINGS

The methods will now be described by way of exemplary embodiments with reference to the accompanying simplified, diagrammatic, not-to-scale drawings:

FIG. 1 is a general schematic of a typical end pit lake during the winter season comprising FFT and a water cap.

FIG. 2 shows photographs of three ice cores that were obtained at three distinct locations of one of the applicant's end pit lakes.

FIG. 3A shows a CT scan of an ice comprising gas bubbles where some of the gas bubbles are coated with bitumen.

FIG. 3B shows two graphs that illustrate the bubble size distribution throughout the ice core that can be determined from the CT scans of FIG. 3A.

FIG. 4A is a photograph of a pail containing a melted ice core which was shown to be comprised of essentially clear water with a trace of fine solids at the bottom of the pail.

FIG. 4B is a photograph of a pail containing an ice core that was retrieved over an area of an end pit lake that was known to contain bitumen mats at its mudline.

FIG. 5 shows a bitumen flux contour plot obtained from ice core data of an isolated area of one of the applicant's end pit lakes.

FIG. 6 shows the absorbance spectra for ice core samples taken from an end pit lake and ice core samples taken from a fresh water lake, where absorbance is on the y-axis and wavelength is on the x-axis.

### DETAILED DESCRIPTION

The detailed description set forth below in connection with the appended drawings is intended as a description of various embodiments of the methods herein and is not intended to represent the only embodiments contemplated. The detailed description includes specific details for the purpose of providing a comprehensive understanding of the present methods. However, it will be apparent to those skilled in the art that the present methods may be practised without these specific details.

It was discovered by the present applicant that the hydrocarbon sheen on the surface of end pit lakes is a result of the existence of sunken bitumen, also referred to herein as sunken bitumen mats. Initially, because the oil sand tailings still contain a small amount of bitumen, when the tailings are first deposited into an end pit lake, there is significant aeration occurring that causes a very small portion of the bitumen droplets present in the tailings stream to attach to air bubbles. These aerated bitumen droplets then float to the surface of the water to form a bitumen mat on the surface of the lake. Over time, these floating bitumen mats deaerate and become denser than water. As a result, the mats sink to the interface of the tailings and water, or mudline.

Research has shown that methane bubbles may be formed below these bitumen mats as a result of microbial activity, e.g., by methanogens, primarily due to the presence of residual naphtha from froth treatment tailings, which is being broken down to form methane bubbles. The methane bubbles then rise to the surface of the end pit lake and are coated with bitumen when travelling through the bitumen mats. These bitumen coated bubbles reach the surface of the end pit lake and burst, leaving a small amount of bitumen on the water surface that eventually spreads into a thin sheen. In addition, methane bubbles can also originate from the bitumen mats themselves, whereby the methane bubbles are coated with bitumen and then rise to the water surface to form the sheen.

Given that gas bubbling will likely continue for a period of time, stopping the flux of bitumen to the surface will require that these bitumen mats be removed by a dredging operation. Thus, acceptance of these end pit lakes as a viable reclamation technology for FFT will require that the transport of bitumen from sunken bitumen mats to the surface of the lake be monitored so that the appropriate intervention of the formation of hydrocarbon sheen can be implemented.

As used herein, “oil sands tailings” mean tailings derived from an oil sands extraction process and include fluid fine tailings (FFT) from tailings ponds and fine tailings from ongoing extraction operations (for example, flotation tailings, thickener underflow or froth treatment tailings) which may or may not bypass a tailings pond.

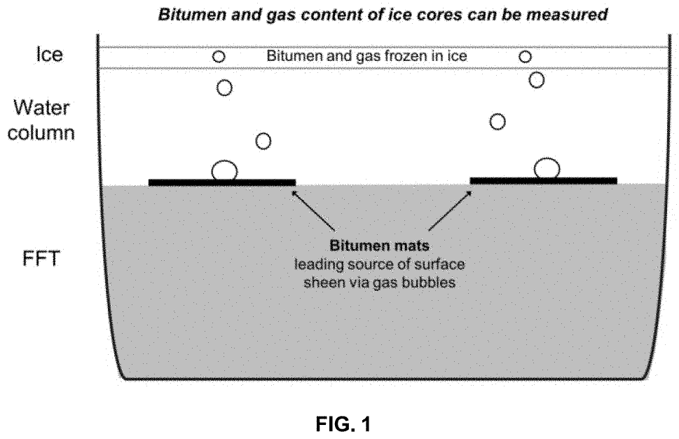

FIG. 1 is a general schematic of a typical end pit lake during the winter season comprising FFT and a water cap. The end pit lake settles and forms a bottom layer of FFT, a top layer of water (i.e., water column) and an interface between the FFT and water column. As previously discussed, bitumen mats may form at the interface of the FFT and water column and it is believed that these bitumen mats are the leading source of surface sheen via gas bubbles such as methane forming in the FFT. During the winter season, an ice layer forms and in this ice layer are trapped bitumen and gas bubbles. As previously mentioned, because the water column is very stable and calm during the winter, there is a high degree of certainty that what is captured in the ice layer or cap is located directly above where it was released from the interface or mudline.

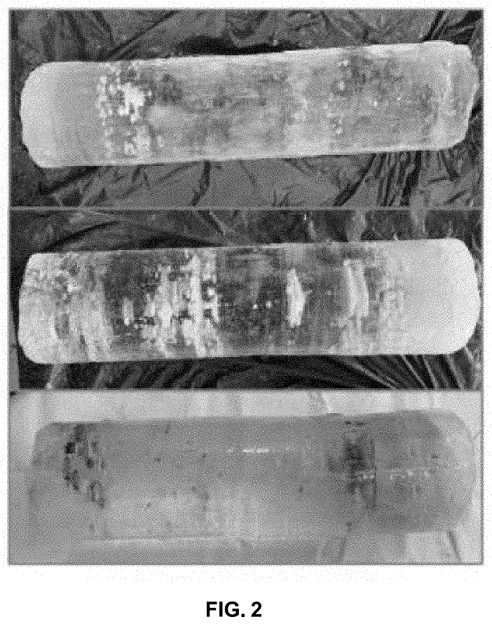

Ice cores can be collected during the winter at various sites along the frozen end pit lake. An ice coring device is used to obtain, for example, ice cores have a diameter of about 14 cm and a length of about 0.5 to 0.7 meters. FIG. 2 shows three ice cores that were obtained at three distinct locations of one of the applicant's end pit lakes. It can be seen that the top two ice cores contain varying amounts of bubbles while the bottom core can be seen to have some hydrocarbon associated with some of the bubbles therein.

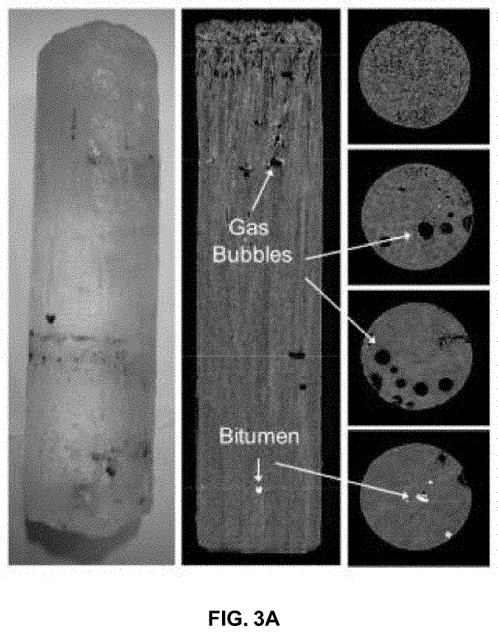

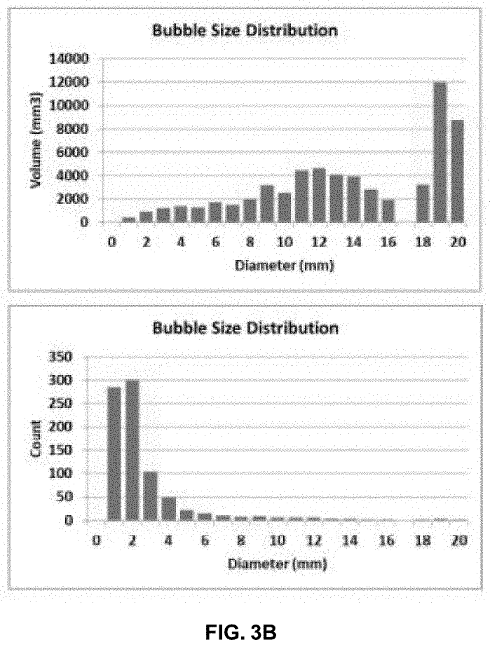

FIG. 3A shows a CT scan of an ice comprising gas bubbles where some of the gas bubbles are coated with bitumen. The left hand photograph in FIG. 3A is of the ice core itself prior to scanning. The middle photograph is a CT scan of the entire length of the ice core. Gas bubbles appear as dark (black) solid circles and the bitumen coated bubbles appear as white solid circles. The right hand photographs are cross sectional CT scans of the ice core, once again where the gas bubbles appear as dark (black) solid circles and the bitumen coated bubbles appear as white circles. Thus, by using a CT scanner, one can determine the volume of gas in a given ice core and can even detect small accumulations of bitumen.

FIG. 3B shows two graphs that show the bubble size distribution throughout the ice core that can be determined from the CT scan. The top graph shows the bubble size distribution of the bubbles (diameter (mm)) (y-axis) versus the volume (mm³) of the ice core (x-axis). The bottom graph shows the number of bubbles (count) (x-axis) versus the size of the bubbles (diameter (mm)) (y-axis). It can be seen that CT scans of ice cores can detect both the amount of bubbles in the ice core and the size of the bubbles in the ice core.

Thus, for this particular ice core, the majority of the gas volume is contained in a few larger bubbles of around 20 mm in diameter; however, it can be seen that the predominant bubble size is in the order of around 1 mm in diameter.

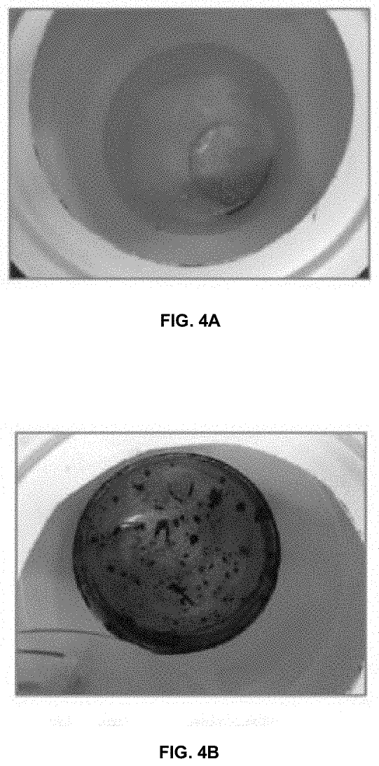

Once the CT scans on the ice cores are complete, the amount of bitumen in each ice core is determined by using ice core melting pails to melt the ice cores while making sure the entire volume of water is retained. FIG. 4A shows a pail containing a melted ice core which was shown to be comprised of essentially clear water with a trace of fine solids at the bottom of the pail. FIG. 4B shows a pail containing an ice core that was retrieved over an area of the lake that was known to contain bitumen mats at its mudline. It can be seen in FIG. 4B that there is a significant amount of bitumen floating on the surface of the water. The bitumen content in each ice core was determined once the ice cores were melted by using the Dean & Stark Soxhlet extraction method that is well known in the art. In particular, after the ice cores were fully melted, each ice core was passed through a Dean & Stark extraction thimble, whereby the bitumen droplets and solids are retained in the thimble and the bulk of the melted ice core water passing through the thimble into a separate pail, to be determined gravimetrically. Only the portion of the melted ice core that was retained on the thimble was loaded into a Dean & Stark extractor for toluene extraction (using 1 L of toluene). Two methods were used to measure the hydrocarbon concentration in each toluene extraction, namely, a Dean & Stark gravimetric based method (where 5 mL of a 0.45 micron filtered toluene extract is pipetted onto a pre-weighed filter paper, dried of toluene in a fume hood, and then re-weighed to determine the bitumen content) and a colorimetry based method (where a relationship is established between the dissolved bitumen content in toluene with the toluene solution's absorbance at a suitable wavelength such as 520 nm, or, optionally, at 420 nm for better sensitivity). It was determined that the colorimetry based method was more accurate at lower concentrations of hydrocarbon, e.g., less than 2.5 g/L.

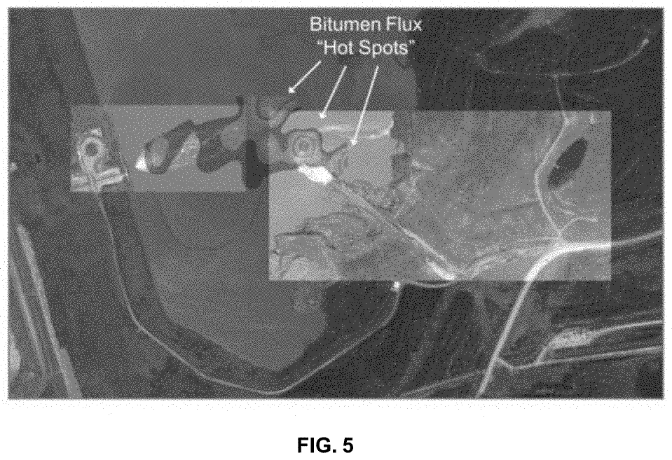

Thus, the bitumen data obtained from ice core analyses can be used to generate a bitumen flux contour map in order to determine the intensity of bitumen flux within a location. FIG. 5 shows a bitumen flux contour plot obtained from ice core data of an isolated area of one of the applicant's end pit lakes, whereby the bitumen flux intensity goes from Blue (low) to red (high), where the red colour denotes the “hot spots”. Also shown in FIG. 5 are the two tailings inflows that are characterized by white plumes, which plumes show that there is a significant amount of aeration from both inflows. As previously mentioned, aeration would cause a bitumen mat to form on the water surface and then over time the mat would sink. Hence, it can be seen that the bitumen flux “hot spots” directly line up with the inflows.

Once a bitumen flux contour plot has been generated from ice core data, the “hot spots”, i.e., the high areas of bitumen flux, will indicate which areas of the end pit lake should be dredged to prevent the formation of hydrocarbon sheen on the surface of the lake. Once the hot spots have been dredged, ice cores can be obtained from these areas in the winter and analysed to see how effective the dredging process was.

One method that could be used to determine the effectiveness of the dredging process is to use Ultraviolet-Visible absorbance spectra (hereinafter referred to as “UV-Vis spectra”). In particular, ice cores can be collected from areas of the frozen end pit lake where the bitumen has been dredged and then the ice cores UV-Vis spectra can be compared to the UV-Vis spectra of ice cores obtained from a fresh water lake or the like. Much the same as with the Dean Stark method for determining hydrocarbon concentrations, toluene extracts (e.g., using 1 L of toluene) of the various ice cores (melted) can be used to determine the UV-Vis spectra of each of the ice cores using a Varian/Cary 5000 UV-Vis-NIR spectrophotometer. As a comparison, the UV-Vis spectra of Royalty bitumen toluene solutions (0.0055 g/L and 0.1 g/L) are used.

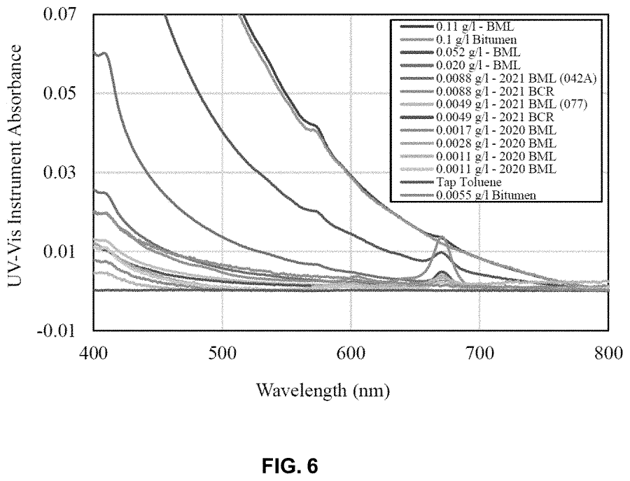

The UV-Vis spectra from various ice cores of an end pit lake (referred to as “BML”) can be seen in FIG. 6. The various hydrocarbon concentrations of each BML ice core are also given, as well as the year the ice cores were obtained, i.e., either 2020 or 2021. FIG. 6 also shows the UV-Vis spectra from two ice cores obtained from a fresh water lake (referred to as “BCR”), where the hydrocarbon concentrations of the BCR ice cores and the year the ice core were obtained are also given. It is believed that the hydrocarbon concentrations in the fresh water lake ice core samples are due to organic material that is normally found in fresh water bodies such as decomposition products of plant material, bacteria, algae, phytoplankton, zooplankton, and the like. Two Royalty bitumen toluene solutions (labelled 0.0055 g/L Bitumen and 0.1 g/L Bitumen) were used as standards, as well as toluene alone (referred to as Tap Toluene).

It can be seen from FIG. 6 that a number of end pit lake ice core samples (e.g., 0.0011 g/L-2020 BML, 0.0028 g/L-2020 BML, 0.0017 g/L-2020 BML, 0.0011 g/L-2020 BML, and 0.0049 g/L-2021 BML (077)) gave similar UV-Vis spectra as the two ice core samples from the fresh water lake (BCR), both of which were obtained in 2021 (i.e., 0.0088 g/L-2021 BCR and 0.0049 g/L-2021 BCR). In particular, all of the BML ice core extracts with hydrocarbon concentrations equal to or less than the BCR ice core extracts have very similar absorbance spectra to the BCR absorbance spectra. One distinguishing feature of the BML and BCR extracts when compared to the Royal bitumen solutions (0.0055 g/L and 0.1 g/L) is the bump in the spectra at around 670 nm, which is characteristic of phytoplankton (see Ciotti, Aurea M. et al., Assessment of the relationships between dominant cell size in natural phytoplankton communities and the spectral shape of the absorption coefficient, Limnol. Oceanogr., 47(2), 2002, p. 404-417). This suggests that the end pit lake (BML) ice core samples were able to support aquatic life such as phytoplankton.

In summary, ice coring can be used to proactively determine bitumen “hot spots”, i.e., location of sunken bitumen mats, in order to implement the dredging process. Ice coring can then be used to determine the effectiveness of the dredging process. In addition, ice coring can be used to track the longer-term evolution of the gas and bitumen dynamics of the lake. By generating quantitative data of gas and bitumen flux to the surface of an end pit lake, a viable hydrocarbon mitigation strategy can be developed. Effectiveness of a hydrocarbon mitigation strategy can be monitored in several ways, including by obtaining further ice cores and comparing the UV-Vis spectra of these ice cores with ice cores obtained from a fresh water body such as a fresh water lake.

References in the specification to “one embodiment”, “an embodiment”, etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to affect or connect such module, aspect, feature, structure, or characteristic with other embodiments, whether or not explicitly described. In other words, any module, element or feature may be combined with any other element or feature in different embodiments, unless there is an obvious or inherent incompatibility, or it is specifically excluded.

It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation. The terms “preferably,” “preferred,” “prefer,” “optionally,” “may,” and similar terms are used to indicate that an item, condition or step being referred to is an optional (not required) feature of the invention.

The singular forms “a,” “an,” and “the” include the plural reference unless the context clearly dictates otherwise. The term “and/or” means any one of the items, any combination of the items, or all of the items with which this term is associated. The phrase “one or more” is readily understood by one of skill in the art, particularly when read in context of its usage.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values and ranges proximate to the recited range that are equivalent in terms of the functionality of the composition, or the embodiment.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc.

As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio.

What is claimed is:

1. A method for determining a source of a surface bitumen sheen in an end pit lake formed by water capping of oil sand tailings, comprising:
using an ice coring device to obtain an ice core sample having an area from the end pit lake;
melting the ice core and measuring a bitumen content in grams in the ice core sample; and
quantifying a bitumen flux by dividing the grams of bitumen by the area of the ice core divided by a time period between initial ice formation and the time the ice core was obtained.

2. The method as claimed in claim 1, further comprising quantifying a gas content of the ice core in milliliters using a computed tomography (CT) system and measuring a gas flux in the end pit lake for monitoring microbial activity in the end pit lake over time, whereby the gas flux is determined by the milliliters of gas divided by the area of the core divided by the time period between initial ice formation and the time the ice core was obtained.

3. The method as claim in claim 2, wherein the CT system comprises a medical CT scanner.

4. The method as claimed in claim 1, wherein the bitumen content in the ice core is measured by melting the ice core and using the Dean & Stark Soxhlet extraction method to measure the grams of bitumen.

5. The method as claimed in claim 1, wherein the amount of bitumen is measured as grams of bitumen per ice core area.

6. The method as claimed in claim 4, wherein the melted ice core is passed through a Dean & Stark extraction thimble such that the bitumen droplets and solids are retained in the thimble and the bitumen content in the thimble is measured by the Dean & Stark Soxhlet extraction method, and the melted ice core water that passes through the thimble is collected in a separate container to be optionally measured gravimetrically.

7. The method as claimed in claim 1, whereby the oil sand tailings comprises fluid fine tailings (FFT).

8. A method for evaluating end pit lake reclamation, comprising:
using an ice coring device to obtain an ice core sample from the end pit lake and an ice core sample from a fresh water body;
melting the end pit lake and fresh water body ice cores and extracting the hydrocarbons therein using toluene to obtain an end pit lake toluene extract and a fresh water body toluene extract;
determining an Ultraviolet-Visible absorbance spectrum of the end pit lake toluene extract and an Ultraviolet-Visible absorbance spectrum of the fresh water body toluene extract; and
comparing the two spectra,
whereby, when the end pit lake spectrum is substantially the same as the fresh water spectrum, it is an indication that the end pit lake is developing the capability to support aquatic life forms.

9. The method as claimed in claim 8, wherein the two spectra are compared to determine whether there is a peak at a wavelength of 670 nm in each of the spectra indicating the presence of plankton.
